(19) United States
(12) Patent Application Publication
(10) Pub. No.: US 2023/0386626 A1
(43) Pub. Date: Nov. 30, 2023

(54) MEDICAL RECORD GENERATION PLATFORM

(71) Applicant: Augmedix Operating Corporation, San Francisco, CA (US)

(72) Inventors: Sarah Rocio NIEHAUS, Ross, CA (US); Sandra BREBER, Orinda, CA (US); Nathanael WOLFE, Bozeman, MT (US); Davin LUNDQUIST, Camarillo, CA (US)

(73) Assignee: Augmedix Operating Corporation, San Francisco, CA (US)

(21) Appl. No.: 18/310,816

(22) Filed: May 2, 2023

Related U.S. Application Data
(60) Provisional application No. 63/345,403, filed on May 24, 2022.

(51) Int. Cl. G16H 10/60; G16H 15/00

(52) U.S. Cl. CPC G16H 10/60; G16H 15/00

(57) ABSTRACT

Systems and methods for generating and displaying medical records, such as a medical note, are provided. The systems and methods include displaying a graphical user interface comprising a plurality of screens; receiving transcript data of a medical consultation; generating one or more medical indicators based on the transcript data of the medical consultation; displaying, on a first screen of the plurality of screens, a plurality of medical indicators including the one or more medical indicators generated based on the received transcript data; receiving a user input; updating at least one medical indicator of the plurality of medical indicators displayed on the first screen based at least in part on the received user input; generating at least a portion of the medical note based on information indicated by the updated at least one medical indicator; and displaying, on a second screen of the plurality of screens, the generated medical note.

### CROSS-REFERENCE TO RELATED APPLICATIONS

This application claims benefit of priority of U.S. Provisional Patent Application No. 63/345,403, filed May 24^{th}, 2022, the entire contents of which are hereby incorporated by reference.

### FIELD

This disclosure relates generally to electronic health record systems, and more specifically to systems, methods, and graphical user interfaces for generating and displaying medical notes for electronic health records.

### BACKGROUND

Electronic health records are vital in providing, documenting, and tracking medical care across all medical fields and specialties. According to known techniques, medical practitioners and medical records specialists (e.g., scribes) manually write medical notes describing consultations with patients in order to record the patient's demographic information, prior medical information, previously prescribed medication information, complaint and symptom information, and information regarding any treatment, tests, or medication prescribed for the patient during the consultation. The handwritten notes are later translated into the electronic health record for the patient.

### SUMMARY

As described above, medical notes regarding consultations with patients are created for electronic health records by being manually written by a medical practitioner and/or medical record specialist. However, said techniques are time-consuming and labor-intensive. Furthermore, manually creating medical notes is prone to human error and may produce medical notes that are not in any standardized format and thus are poorly suited for future manual and/or automated review/analysis.

Disclosed herein are systems, methods, platforms, and graphical user interfaces that may improve the creation of medical records, such as medical notes, stored in electronic health records (EHRs). Notably, a computerized system may receive audio conversation data, transcript conversation data, and/or electronic medical record (EMR) data comprising medical information from a medical consultation between a medical practitioner and patient. For example, the medical information may pertain to any aspect of patient medical information, such as a symptom, onset mode of a symptom, onset timing of a symptom, frequency (e.g., of a symptom), location of a symptom, contextual information, reason for the visit, quality of a symptom, a prior medical condition, a current medication, a medication to be prescribed, a treatment to be prescribed, lab test results, a lab test to be ordered, imaging procedure results, an imaging procedure to be ordered, an organ system, a diagnostic procedure, a diagnosis, a treatment, etc.

Based on the received conversation data, the system may automatically populate one or more fields of a graphical user interface (GUI) with medical indicators based on medical entities extracted from the conversation data. In some embodiments, a user may modify (e.g., activate, add, delete, etc.) one or more medical indicators. The modification by the user may be received as a user input that may comprise additional medical information related to the medical consultation. The system may generate and display a structured medical note based at least on the active medical indicators on a graphical user interface (GUI). The medical note may comprise complete sentences, arranged in paragraph form, that are automatically generated by the system. The systems and methods provided herein may produce medical notes in a manner that is more efficient, resistant to user error, flexible, configurable, and scalable than traditional written medical record creation. The use of the systems, methods, platforms, and interfaces described herein may extend to other types of medical records, such as medical orders (e.g., lab tests, prescriptions, etc.), medical coding, after-visit summaries, care reminders, pre-charting summaries, etc. The systems, methods, and GUIs disclosed herein may display and store medical records in a consistent, structured format such that the medical notes may be efficiently and accurately reviewed and analyzed (whether manually or programmatically) after creation and storage.

In some embodiments, a system for generating and displaying a medical note is provided, the system comprising one or more processors configured to cause the system to: display a graphical user interface comprising a plurality of screens; receive transcript data of a medical consultation; generate one or more medical indicators based on the received transcript data of the medical consultation; display, on a first screen of the plurality of screens, a plurality of medical indicators including the one or more medical indicators generated based on the received transcript data; receive a first user input; update at least one medical indicator of the plurality of medical indicators displayed on the first screen based at least in part on the received first user input; generate at least a portion of a medical note based on information indicated by the updated at least one medical indicator; and display, on a second screen of the plurality of screens, the generated medical note.

In some embodiments, a state of the at least one medical indicator is interchangeable between a plurality of states comprising two or more states of the following: a confirmed present state, a confirmed absent state, a suggested present state, and a suggested absent state.

In some embodiments, updating the at least one medical indicator comprises changing the state of the at least one medical indicator based on the patient medical information.

In some embodiments, the state of the at least one medical indicator is updated to the confirmed present state or confirmed absent state.

In some embodiments, displaying the plurality of medical indicators comprises, in accordance with a given medical indicator of the plurality of medical indicators having been generated based on the received transcript data, displaying the given medical indicator in a state selected from the following: the suggested present state and the suggested absent state.

In some embodiments, the one or more processors are configured to cause the system to, in accordance with not receiving a user input comprising an instruction to update the given medical indicator, generate at least a portion of the medical note to include information indicated by the given medical indicator.

In some embodiments, the one or more processors are configured to cause the system to generate one or more medical indicators included in the displayed plurality of medical indicators based on stored template data; wherein displaying the plurality of medical indicators comprises, in accordance with a given medical indicator of the plurality of medical indicators having been generated based on the stored template data, displaying the given medical indicator in a state selected from the following: the suggested present state and the suggested absent state.

In some embodiments, the one or more processors are configured to cause the system to, in accordance with not receiving a user input comprising an instruction to update the given medical indicator, generate at least a portion of the medical note without inclusion of information indicated by the given medical indicator.

In some embodiments, updating the at least one medical indicator comprises adding the at least one medical indicator based on the patient medical information.

In some embodiments, the one or more processors are configured to cause the system to automatically organize the plurality of medical indicators displayed on the first screen based on at least complaint type and medical note sections, wherein the medical note sections comprise two or more sections of the following: history of present illness, physical examination, review of systems, and assessment/plan.

In some embodiments, the one or more processors are configured to cause the system to automatically organize the plurality of medical indicators in a given medical note section displayed on the first screen based on one or more sub-sections of the following: medications, symptoms, treatments, tests, and assessments.

In some embodiments, the one or more processors are configured to cause the system to display, on a third screen of the plurality of screens, a representation of the received transcript data.

In some embodiments, the one or more processors are configured to cause the system to indicate one or more of the generated medical indicators in the representation of the transcript data on the third screen.

In some embodiments, indicating the one or more of the generated medical indicators in the representation of the transcript data comprises displaying a portion of text in the representation of the transcript data in a text color that differs from other text in the representation of the transcript data, wherein the portion of text corresponds to the one or more of the generated medical indicators.

In some embodiments, the one or more processors are configured to cause the system to display, on a fourth screen of the plurality of screens, a plurality of graphical representations of respective medical consultations for a medical professional.

In some embodiments, each graphical representation of a medical consultation indicates a respective medical note status for the medical consultation.

In some embodiments, the one or more processors are configured to cause the system to, while displaying the fourth screen, receive a second user input comprising an indication of a selection of a graphical representation of a respective medical consultation of the plurality of graphical representations of respective medical consultations.

In some embodiments, the one or more processors are configured to cause the system to responsively display the first screen of the plurality of screens on the graphical user interface based on the second user input.

In some embodiments, receiving the first user input comprising patient medical information comprises receiving an indication of a selection from a displayed list of options.

In some embodiments, one or more processors are configured to cause the system to, while displaying the second screen, receive a third user input comprising medical information.

In some embodiments, receiving the third user input comprises one or more of the following: receiving entry of text into one or more text fields, receiving audio dictation, and receiving a selection from a displayed list of options.

In some embodiments, the graphical user interface is embodied in a mobile user interface and at least one of the plurality of screens are touch-sensitive screens.

In some embodiments, the one or more processors are configured to cause the system to store the medical note in an electronic health record (EHR) of a patient.

In some embodiments, a method for generating and displaying a medical note is provided, the method performed at a system comprising one or more processors, the method comprising: displaying a graphical user interface comprising a plurality of screens; receiving transcript data of a medical consultation; generating one or more medical indicators based on the received transcript data of the medical consultation; displaying, on a first screen of the plurality of screens, a plurality of medical indicators including the one or more medical indicators generated based on the received transcript data; receiving a first user input; updating at least one medical indicator of the plurality of medical indicators displayed on the first screen based at least in part on the received user input; generating at least a portion of a medical note based on information indicated by the updated at least one medical indicator; and displaying, on a second screen of the plurality of screens, the generated medical note.

In some embodiments, a non-transitory computer-readable storage medium storing instructions for generating and displaying a medical note is provided, the instructions configured to be executed by a system comprising one or more processors to cause the system to: display a graphical user interface comprising a plurality of screens; receive transcript data of a medical consultation; generate one or more medical indicators based on the received transcript data of the medical consultation; display, on a first screen of the plurality of screens, a plurality of medical indicators including the one or more medical indicators generated based on the received transcript data; receive a first user input; update at least one medical indicator of the plurality of medical indicators displayed on the first screen based at least in part on the received first user input; generate at least a portion of a medical note based on information indicated by the updated at least one medical indicator; and display, on a second screen of the plurality of screens, the generated medical note.

### BRIEF DESCRIPTION OF THE FIGURES

Various embodiments are described with reference to the accompanying figures, in which:

FIG. 1 depicts a system for providing a medical record generation platform, in accordance with some embodiments.

FIGS. 2A-2P depict screens of a graphical user interface (GUI) for generating and displaying medical records, in accordance with some embodiments.

FIG. 3 depicts a method diagram for generating and displaying medical records, in accordance with some embodiments.

FIG. 4 depicts a computer for generating medical records, in accordance with some embodiments.

### DETAILED DESCRIPTION

As described above and in further detail below, the disclosure herein pertains to various systems, methods, computer-readable storage media, platforms, mobile user interfaces, and/or graphical user interfaces for automatically generating medical records (e.g., medical notes) for electronic health records. Traditional medical note generation techniques are time-intensive and laborious for medical practitioners and/or medical records specialists. Additionally, the generated medical notes are often structured in an inconsistent manner and are prone to human error. The computerized system described herein may provide a front-end graphical user interface (GUI) configured to be used by a medical practitioner to record patient medical information, for example regarding a medical consultation with a patient. The medical practitioner may input and/or retrieve data, such as audio conversation data, transcript conversation data, and/or existing medical records comprising patient medical information to the system. The conversation data may comprise transcript and/or audio data of one or more individuals. The system may automatically and systematically generate an output, such as a medical note, based at least on the input data. The medical note may be generated in a structured manner such that the record may be easily accessible for later review and analysis (both manually and programmatically).

The systems described herein may be described primarily with respect to a particular type of medical record output, such as a medical note. However, it is to be understood that the systems may be configured to generate various types of medical record outputs, including but not limited to medical billing codes, prescription/test orders, pre-charting summaries, after-visit care summaries, care reminders, etc.

The system may be provided, in some embodiments, as locally hosted software and/or by one or more servers providing the platform and graphical user interface via a network system (e.g., by providing a GUI as part of a dedicated program/application and/or through a web-browser interface). The graphical user interface may include a plurality of screens, wherein one or more of the screens comprises a plurality of graphical objects (e.g., GUI objects, such as selectable icons, buttons, labels, symbols, etc.) representing medical indicators that may pre-populate based at least in part on the processed conversation data. In some embodiments, the graphical user interface may be embodied in a mobile user interface comprising a touch-sensitive screen. The user may engage with the medical indicators, for example, to modify (e.g., add, delete, activate, etc.) the state of medical indicators, thus confirming medical information of the patient. Patient medical information may include, for example, medical information pertaining to any aspect of patient medical information, such as a symptom, onset mode of a symptom, onset timing of a symptom, frequency (e.g., of a symptom), location of a symptom, contextual information, reason for the visit, quality of a symptom, a prior medical condition, a current medication, a medication to be prescribed, a treatment to be prescribed, lab test results, a lab test to be ordered, imaging procedure results, an imaging procedure to be ordered, an organ system, a diagnostic procedure, a diagnosis, a treatment, etc.

### Medical Records Generation Engine

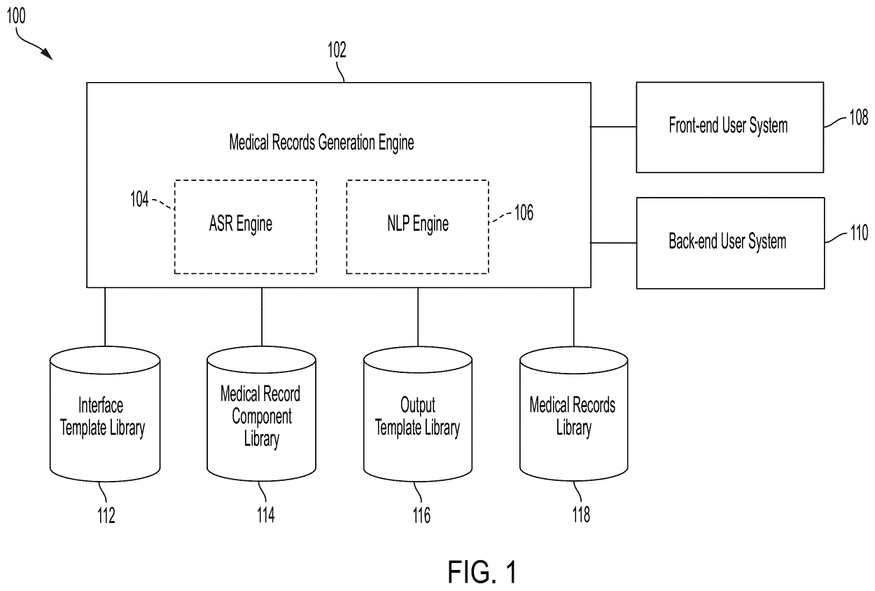

Systems for generating and displaying medical notes based at least on audio and/or transcript conversation data may comprise one or more engines comprising one or more processors communicatively coupled with one or more libraries, front-end user systems, and/or back-end user systems. FIG. 1 illustrates a system 100 for providing a medical record generation platform, in accordance with some embodiments.

As shown in FIG. 1, system 100 may include medical records generation engine 102, which may include an automatic speech recognition (ASR) engine 104 and a natural language processing (NLP) engine 106. In some embodiments, engines 104 and 106 may be provided by a single processor or a common set of processors. In some embodiments, engines 104 and 106 may be provided by different processors or different sets of processors. In some embodiments, medical records generation engine 102 may be provided by one or more processors aside from the processors providing engines 104 and 106. System 100 may also include front-end user system 108, back-end user system 110, interface template library 112, medical record component library 114, output template library 116, and medical records library 118. As shown, each of the components of system 100 may be communicatively coupled (e.g., by wired and/or wireless electronic communication) with engine 102, such that one or more processors of engine 102 may facilitate the receipt and/or transmission of data between libraries and/or systems (e.g., front-end user system 108, back-end user system 110, etc.). In some embodiments, system 100 may be provided as a distributed (e.g., network) system with one or more components located remotely from one another and connected via network (e.g., wide-area network) communication. In some embodiments, system 100 may be provided as a local system with one or more components located together with one another and connected via local network communication. In some embodiments, one or more components of system 100 may be provided as part of a single computer device. In some embodiments, system 100 may provide a platform by which a front-end user of system 108 may be provided with one or more graphical user interfaces (GUIs) to generate and store medical notes for electronic health records. In some embodiments, a front-end user of front-end user system 108 may be provided with one or more mobile user interfaces comprising a touch-sensitive screen and one or more GUIs.

Medical records generation engine 102 may comprise any one or more processors (located locally and/or remotely from front-end system 108 and/or back-end system 110) configured to perform all or part of any of the techniques disclosed herein. In some embodiments, engine 102 may be provided, in whole or in part, as one or more processors of a personal computer, laptop computer, tablet, mobile electronic device, server, distributed computing system, and/or cloud computing system.

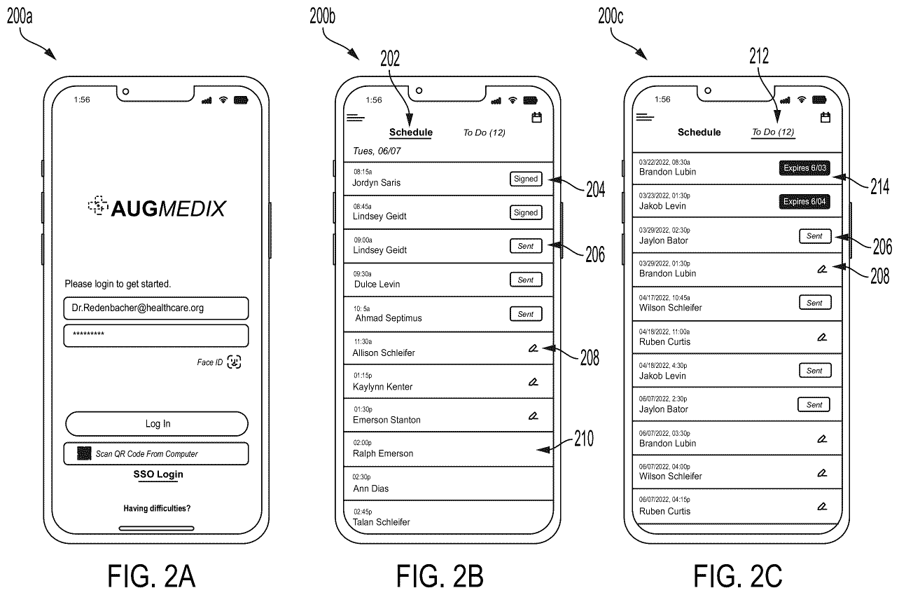

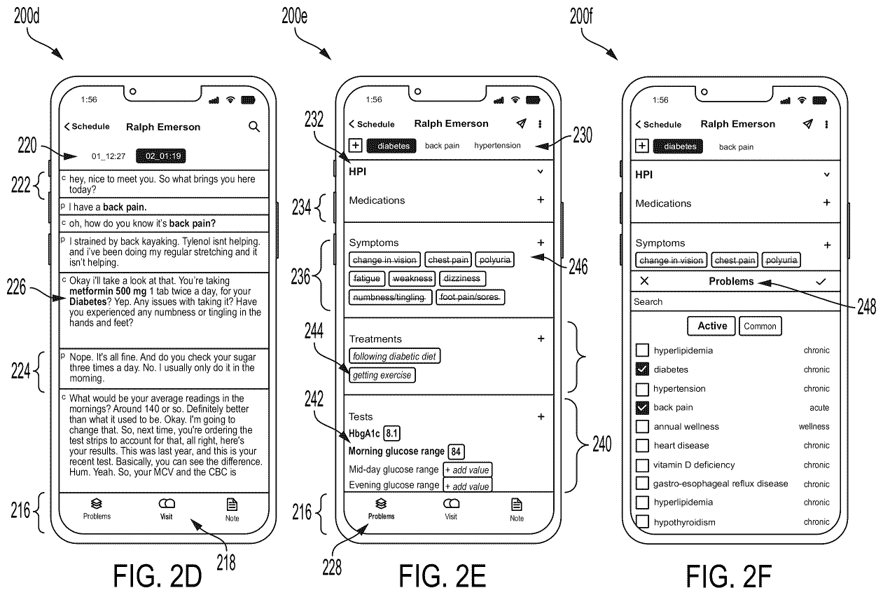

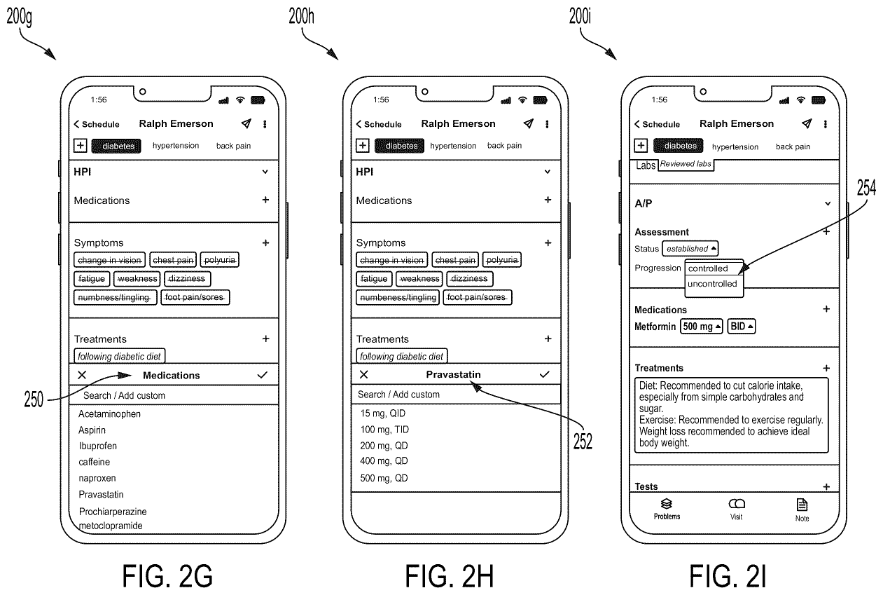

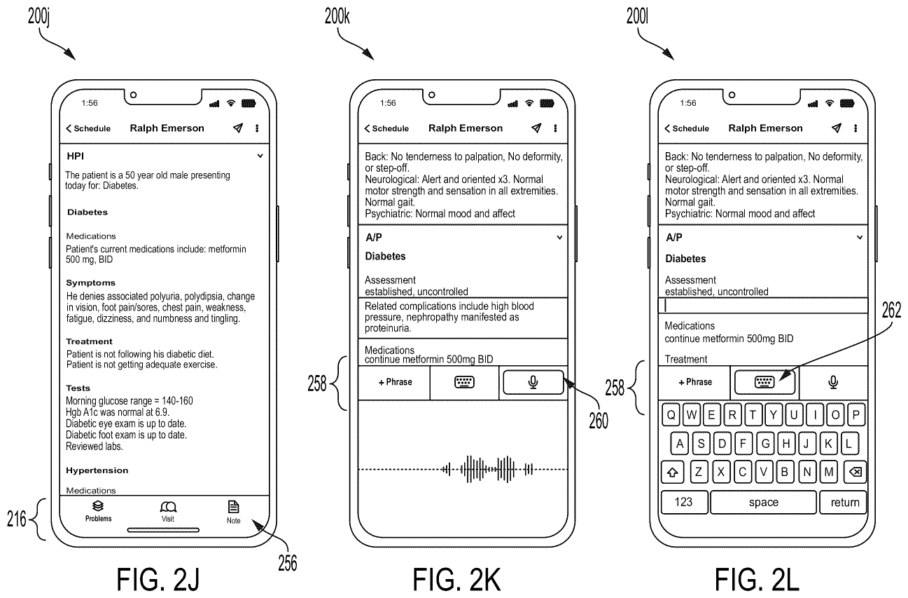

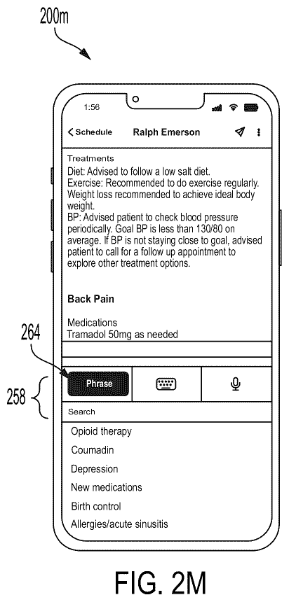

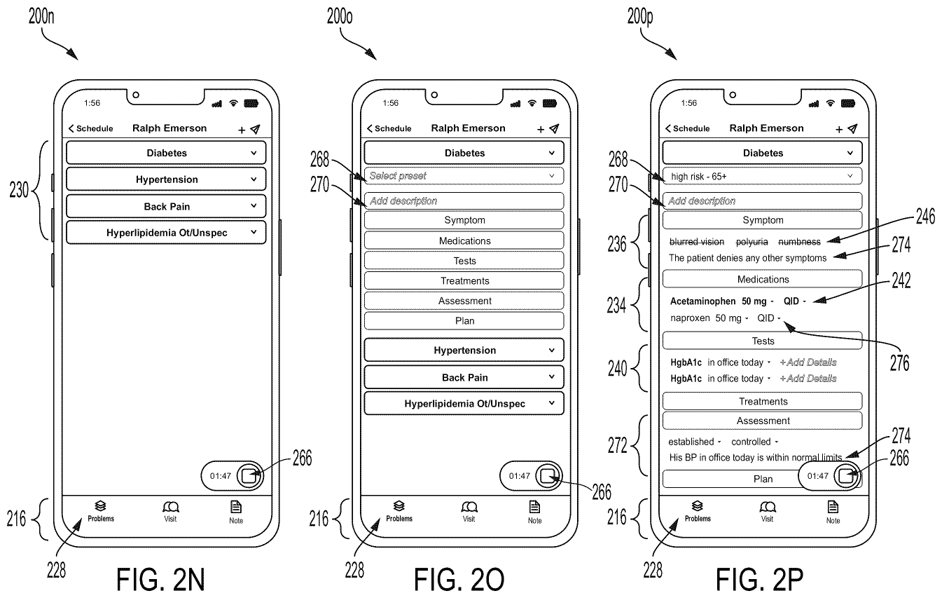

Engine 102 may be configured to provide one or more graphical user interfaces (e.g., the interface described below with respect to FIGS. 2A-2P) to front-end users of the system such that the front-end users may supply information to system 100 regarding a medical consultation with a patient. In some embodiments, the graphical user interface may be embodied in a mobile user interface with a touch-sensitive screen. Engine 102 may provide instructions for providing one or more graphical user interface screens to front-end user system 108 such that system 108 may display a graphical user interface and receive user inputs via said graphical user interface.

Engine 102 may then receive (e.g., via wired or wireless electronic transmission) data transmitted from front-end user system 108 regarding the user inputs detected by system 108. Based on the data received regarding the front-end user inputs, engine 102 may generate a medical note, wherein the medical note may describe one or more aspects of the medical consultation corresponding to the provided front-end user inputs. In some embodiments, the system may receive user inputs comprising audio data for processing from any suitable source (e.g., front-end user system 108). For example, a user interface provided by front-end system 108 may provide users the opportunity to upload audio data, transcript data, and/or use a personal computing device (e.g., mobile device, workstation, desktop, tablet, etc.) comprising a microphone to capture raw audio data in real-time, and the audio and/or transcript data may then be transmitted from front-end system 108 to engine 102 for processing.

The medical note may describe patient demographic information, patient background information, patient medical/family hi story information, patient complaint information, patient symptom information, patient preexisting/past medication information, patient preexisting/past treatment information, medication prescription information, test/analysis prescription information, and/or treatment prescription information. In some embodiments, the system may be configured to generate individual natural language phrases, sentences, and/or paragraphs using a natural language phrase structure, sentence structure, and/or paragraph structure accessible to engine 102 (e.g., stored as part of a template structure in output template library 116) for entry into the medical note. Once the medical note is generated, it may be stored (e.g., as part of an electronic health record in medical records library 118) and/or displayed to a user (e.g., by being transmitted to front-end user system 108 for display on a screen).

Front-end user system 108 may comprise any one or more computer systems (located locally and/or remotely from engine 102) configured to receive instructions and/or transmitted data from engine 102, to render and/or display a graphical user interface to a front-end user, to detect one or more inputs executed against the graphical user interface by the user, and to transmit data regarding detected user inputs to engine 102. In some embodiments, front-end user system 108 may include any suitable display and any suitable input device (e.g., mouse, keyboard, touch-sensitive device, touchscreen, microphone, etc.). In some embodiments, front-end user system 108 may be provided, in whole or in part, as a personal computer, workstation computer, laptop computer, tablet, or mobile electronic device. For example, front-end user system 108 may be provided as a mobile user interface comprising at least a graphical user interface (GUI) and touch-sensitive screen. Example graphical user interfaces of front-end system 108 are described in greater detail below.

Back-end user system 110 may comprise any one or more computer systems (located locally and/or remotely from engine 102) configured to send data to and/or receive data from engine 102. In some embodiments, back-end user system 110 may be configured to send instructions to engine 102 in order to configure the user interface to be provided to front-end system 108, such as by configuring options to be presented to front-end users of the interface (e.g., stored in interface template library 112) and/or configuring sentence structures and/or paragraph structures to be used to create medical notes (e.g., stored in output template library 116). In some embodiments, back-end user system 110 may be configured to receive transmissions from engine 102 regarding monitoring front-end users, system performance, system characteristics, and/or metadata collected based on use of the platform and graphical user interfaces by one or more front-end users. In some embodiments, back-end user system 110 may include any suitable display and any suitable input device (e.g., mouse, keyboard, touch-sensitive device, touchscreen, microphone, etc.). In some embodiments, back-end user system 110 may be provided, in whole or in part, as a personal computer, workstation computer, laptop computer, tablet, or mobile electronic device. In some embodiments, front-end user system 108 and back-end user system 110 may be provided on a shared device and/or may be provided as a package in the same computer system or set of computer systems, such that the front-end user and back-end user may be the same individual. Example back-end user systems are described in greater detail in U.S. patent application Ser. No. 17/313,540, the entire contents of which are hereby incorporated by reference in its entirety.

In some embodiments, medical record component library 114 may comprise any one or more computer-readable storage mediums configured to store component information that may be used in the creation of electronic health records and/or in the creation of templates for use in the systems described herein. For example, medical record component library 114 may store data pertaining to medical specialty information, patient visit type information, patient complaint type information, complaint-element information, descriptor information (e.g., information regarding options that may be selected by users to characterize one or more complaint-elements), treatment information, test information, diagnosis information (e.g., diagnosis code information), imaging information, medications information, and/or health systems information.

In some embodiments, the data stored in medical record component library 114 may be used to create (e.g., may be incorporated into) a template executed by the system to provide a graphical user interface for a front-end user. For example, a template may be configured (e.g., by a back-end user of system 110) to provide a plurality of options to a front-end user for specifying what symptoms are/are not present in a patient, the template stored in interface template library 112. In some embodiments, the options for the template may be populated by being automatically drawn from one or more lists or sets of treatment information stored in medical record component library 114. In some embodiments, a template may populate a set of options based on an entire dataset or an entire data subset from library 114. In some embodiments, a template may populate a set of options based on a selection of specific data items from library 114, such as items specified by a back-end user of system 110 in creating the template.

In some embodiments, interface template library 112 may be configured to store the template data mentioned above. Template data may include data (e.g., one or more data structures) configured to be usable by engine 102 to provide all or part of the contents of a GUI to a user of front-end user device 108. In some embodiments, templates may govern what options are displayed to a front-end user of the system and the manner in which they are displayed to the user. In some embodiments, interface template library 112 may store different interface templates for different use cases, including different medical specialties, different languages, different countries, different regions, different states, different medical facilities, different doctors, different patient characteristics or classes, and/or different complaint types. In some embodiments, a front-end user may select an appropriate interface template based on the nature of the patient consultation (e.g., based on the purpose of the patient visit and/or what the patient's complaint is), and the selected interface template may cause the system to display appropriate and relevant options for such a consultation (e.g., from medical record component library 114).

In some embodiments, templates stored in interface template library 112 may be created, updated/modified, and/or deleted by system 100. In some embodiments, a back-end user of system 110 may create, modify, or delete a template by executing inputs comprising instructions to do so to library 112. In some embodiments, library 112 (e.g., via medical records generation engine 102) may automatically update a template based on metadata collected regarding use of the template by one or more front-end users (e.g., if an option in the template is rarely selected, the option may be deprioritized in the template such that it is present in a less prominent manner (e.g., further down in a list); or, if an option that is not automatically presented in a template is frequently manually added by users of the template, then the option may be added to the template such that it is automatically presented in the future.

In some embodiments, output template library 116 may be configured to store template data related to medical records output (e.g., medical note, pre-charting summary, after-visit summary, etc.) structure and/or natural language statement structure. For example, output template library 116 may govern the manner in which the system generates natural language statements with the user inputs (e.g., medical indicators). In some embodiments, the template data stored in interface template library 112 may be configured to apply one or more natural language and/or medical note templates stored in output template library. In some embodiments, output template library 116 may store different medical record and/or statement templates for different use cases, including different medical specialties, different languages, different countries, different regions, different states, different medical facilities, different doctors, different patient characteristics or classes, and/or different complaint types. The template data stored in output template library 116 may dictate the paragraph structure, section structure, and/or document structure displayed on the graphical user interface (GUI).

In some embodiments, medical records library 118 may comprise any one or more computer-readable storage mediums configured to store medical records, such as an electronic health records (EHR). In some embodiments, medical records stored in library 118 may include medical notes comprising natural language statements (e.g., phrases, sentences, etc.) generated by engine 102 in accordance with one or more of the techniques described herein. In some embodiments, the medical records stored in medical records library 118 may be structured such that portions of a medical consultation may be easily accessed, reviewed, and analyzed as training data, for example.

### Graphical User Interface for Generating and Displaying Medical Records

As mentioned above, system 100 described with respect to FIG. 1 may provide graphical user interfaces embodied in, for example, a mobile user interface comprising a touch-sensitive screen, to automatically generate medical notes based on audio and/or transcript conversation data.

FIGS. 2A-2P depict respective screens 200a-200m of graphical user interface 200 of a medical record generation platform, in accordance with some embodiments. In some embodiments, GUI 200 may be displayed on a screen of a front-end user system 108 of system 100, such that a user of GUI 200 may execute inputs via GUI 200 to cause system 100 to generate a medical record (e.g., medical note) pertaining to a medical consultation with a patient. In some embodiments, GUI 200 of front-end system 108 may be configured to receive audio conversation data and/or transcript conversation data inputs. Medical records generation engine 102 may be configured to determine one or more complaints (e.g., problems) and medical indicators related to the complaint(s) to display on one or more screens of GUI 200 based on the conversation data. For example, the medical indicators and/or complaints may correspond to medical entities (e.g., medical words, phrases, and/or sentences) that are extracted from transcript data. The transcript data may be directly uploaded by a user to front-end user system 108 and/or generated by medical records generation engine 102 based on audio conversation data uploaded to and/or recorded at front-end user system 108. Medical records generation engine 102 may be configured to generate a medical note based on the transcript data generated by and/or received at engine 102. In some embodiments, medical records generation engine 102 may be configured to receive data from existing medical records (e.g., retrieved from an EHR) to generate a medical note. Medical records generation engine 102 may additionally or instead be configured to generate a medical note based on the medical indicators activated and/or added by a user on GUI 200.

FIG. 2A depicts screen 200a of graphical user interface 200, in accordance with some embodiments. Screen 200a may depict GUI 200 during the process of signing into a medical record generation system interface (e.g., secure user portal, database, etc.). In some embodiments, a user may sign into the system by manually typing in credentials (e.g., username, email, password, etc.). In some embodiments, a user may sign into the system by using one or more facial recognition tools. In some embodiments, a user may sign into the system by scanning a compatible barcode (e.g., QR code). For example, the medical record generation system may be embodied in a mobile user interface comprising a camera such that the camera may be used to sign into the system (e.g., scanning barcodes and/or facial recognition). The user may select (e.g., click, tap, etc.) an appropriate icon on screen 200a corresponding to the preferred sign-in method to prompt a keyboard to be displayed, activate a camera, etc. In some embodiments, the system may automatically perform a facial recognition based on one or more customizable user preference settings. In some embodiments, a user may choose one or more methods described above to sign into the medical record generation system.

FIG. 2B depicts screen 200b of graphical user interface 200, in accordance with some embodiments. Screen 200b may depict GUI 200 upon signing into the medical record generation system. For example, screen 200b may illustrate a schedule of medical consultations with patients of the user (e.g., medical professional). In some embodiments, the user may toggle screen 200b between a schedule window of medical consultations and a to-do window of medical consultations (illustrated in FIG. 2C) using a menu comprising corresponding GUI objects for the windows. The list of medical consultations provided in a to-do window of GUI 200 may comprise a list of medical consultations associated with medical records, such as notes, that require completion, review, and/or a signature from the user.

The schedule window 202 of screen 200b may comprise a scrollable, list-formatted schedule of medical consultations for the medical professional, each of the medical consultations in the list represented by a selectable GUI object (e.g., a button). In some embodiments, the schedule window 202 may comprise a daily schedule (e.g., the user may view a list of medical consultations scheduled on a given day). In some embodiments, the schedule window 202 may additionally or instead comprise a 3-day or 5-day schedule. In some embodiments, GUI 200 may be customizable such that a user may select the number of days visible in schedule window 202. In some embodiments, the user may toggle between viewing a scrollable list of medical consultations and a calendar view displaying a plurality of days (e.g., one or more weeks, a month, etc.). For example, screen 200a may comprise a selectable icon for toggling between a calendar view and list view of medical consultations, such that the user may tap/click the icon to toggle between views. The user may view selectable GUI objects on a calendar view, each object corresponding to a scheduled medical consultation.

In some embodiments, the GUI objects corresponding to medical consultations may comprise information such as scheduled time of the medical consultation and patient name (e.g., full name, first name, last name, etc.). In some embodiments, GUI objects may additionally include a status of a medical note associated with the medical consultation of the patient, as will be described in greater detail below. In some embodiments, the GUI objects may be sorted in a chronological order, such that the earliest scheduled medical consultations are displayed prior to the later scheduled medical consultations.

In some embodiments, the medical consultations on schedule window 202 of screen 200b may be differentiated based on status of the medical note and/or status of the medical consultation. For example, the appearance, transparency, and/or color of the GUI objects associated with each medical consultation may be differentiated based on the status of the medical consultation. For example, a GUI object may appear transparent when the time of the corresponding medical consultation has passed and/or when the medical note has been generated for the medical consultation. For example, GUI objects 204, 206 illustrate medical consultations associated with a generated medical note. Each of GUI objects 204, 206 may additionally comprise a label indicating the status of the medical note associated with the medical consultation. For example, GUI object 204 comprises a label “signed,” indicating that the medical note of the medical consultation corresponding to GUI object 204 has been signed. GUI object 206 comprises a label “sent,” indicating that the medical note of the medical consultation corresponding to GUI object 206 has been sent to an electronic health record (e.g., medical records library 118). In some embodiments, a medical note may automatically be sent upon the user completing the medical note (e.g., the GUI object may be denoted with a label “sent”), and the system may require the user to access the medical note for review and signature to complete the medical note (e.g., the GUI object may be denoted with a label “signed” upon completion).

In some embodiments, the GUI objects may be differentiated in a manner different from the transparency described above. For example, one or more text portions associated with the GUI object may be highlighted (e.g., bolded, italicized, etc.) to differentiate between statuses of the medical consultation. In some embodiments, the GUI objects may comprise additional or other labels different from those described above for indicating the status of the medical note associated with a medical consultation. For example, the GUI object may comprise color-coding, one or more icons, and/or one or more keywords and/or phrases.

In some embodiments, despite one or more patient GUI objects being signed and/or sent, a user may select (e.g., click/tap) a GUI object and view the complete or incomplete medical note for the medical consultation (e.g., regardless of status of the medical record).

As mentioned above, the platforms described herein may extend to other types of medical records beyond medical notes. Thus, one or more aspects of the schedule window 202 (e.g., the GUI objects) may indicate the type of medical record to be completed. For example, as mentioned above the medical record may comprise pre-visit charting, after visit summary, medical coding, prescription and/or lab test order, etc.

As mentioned above, schedule window 202 of GUI 200 on screen 200b may comprise one or more GUI objects corresponding to medical consultations to be completed (e.g., no medical note generated), medical consultations with partially completed medical notes, and/or medical consultations with completed medical notes (e.g., labeled “sent”). For example, GUI objects 208 and 210 may not be depicted as transparent, in contrast to GUI objects 204 and 206, because the medical notes associated with the medical consultations may require an action of the user. For example, GUI object 208 may correspond to a partially completed medical note of a medical consultation that may require an action from the user to be completed and sent to an EHR. In some embodiments, as shown in FIG. 2B, the status of the medical note may be illustrated by a symbol (e.g., a pencil). The user may select the symbol and/or another part of the GUI object 208 to cause one or more additional screens (described in greater detail below) to be displayed on GUI 200 to complete the medical note. For example, each of the GUI objects shown on screen 200b may be buttons configured to open one or more screens corresponding to the medical note of the medical consultation. GUI object 208 may differ from GUI object 210, for example, in that the medical consultation associated with GUI object 208 may be partially completed. For example, the user may have previously uploaded transcript and/or audio conversation data to the GUI 200 for processing by the system, however, the medical note automatically generated based on the transcript and/or audio conversation data may not yet be completed (e.g., one or more components of the medical note may require additional medical information and/or review, as will be described in greater detail below).

In contrast, the medical consultation associated with GUI object 210 may not yet be associated with any medical record of the medical consultation. Thus, the user may select GUI object 210, for example, and may be prompted to record audio conversation data, upload audio conversation data, and/or upload transcript conversation data. In some embodiments, the user may dictate, type, and/or interact with one or more click point GUI objects to input medical information into a medical note and/or medical note outline, as will be described in greater detail below with respect to FIGS. 2D-2M.

In some embodiments, the GUI objects associated with medical consultations may be filtered such that a portion of the full list of medical consultations may be viewed in schedule window 202. For example, a user may select (e.g., tap/click) an icon on screen 200b and cause one or more filtering settings to be displayed for adding, removing, and/or modifying one or more GUI objects in screen 200b. For example, the user may filter the medical consultations by status of the medical note (e.g., not started, started but incomplete, completed/sent, sent but not yet signed, etc.) and/or status of the medical consultation (e.g., completed/passed, in progress, not yet started/completed, etc.).

As mentioned above, a user may toggle between viewing a schedule of medical consultations and a to-do list related to medical notes, for example by clicking/tapping the GUI objects within a menu region of GUI 200 corresponding with each of the windows. FIG. 2C depicts screen 200c of graphical user interface 200, in accordance with some embodiments. Screen 200c may depict GUI 200 upon signing into the system and/or upon the user toggling from schedule window 202 to to-do window 212. For example, screen 200c may illustrate a list of medical consultations with associated medical notes that may require completion, review, and/or a signature by the user (e.g., a medical professional).

In some embodiments, the GUI objects 206, 208 illustrated in screen 200c of FIG. 2C may comprise any of the features of GUI objects 206, 208 (respectively) described above with respect to FIG. 2B. For example, GUI object 206 may correspond with a medical note of a medical consultation that has been sent to a medical records library but requires a signature from the user. GUI object 206 may be visually transparent on screen 200c. In some embodiments, GUI object 208 may correspond with a medical note of a medical consultation that is yet to be completed.

In some embodiments, the medical consultations displayed in screen 200c of GUI 200 may be organized chronologically in a list format based on the date of the consultation. In some embodiments, as described above with respect to FIG. 2B, the user may toggle between viewing the medical consultations in a list format and in a calendar format. In some embodiments, the user may customize the to-do window 212 to display the medical consultations from a given day or a span of days (e.g., 3 days, 5 days, 1 week, 1 month, etc.).

In some embodiments, medical notes may be required to be sent to an electronic health record (EHR) (e.g., medical records library 118) within a specific duration of time. The system may be configured to indicate a due date for a medical note associated with a medical consultation. For example, GUI object 214 may comprise a label that indicates the date on which the medical note is required to be sent to the HER (e.g., expiration date). In some embodiments, (e.g., dependent upon the proximity of the due date) the label may be color-coded, for example, to indicate the urgency to complete the medical note. For example, if a medical note is required to be completed on the current day, the label may be colored a first color (e.g., red). If a medical note is required to be completed in more than one day, the label may be colored a second color (e.g., yellow). In some embodiments, the number of days associated with the first color and second color of the label may vary. For example, the label associated with a GUI object may be a first color if the medical note is due in the next 3 days, whereas the label may be a second color if the medical note is due in the next week. The label may be customized and/or based on the selected interface template (e.g., stored in interface template library 112).

In some embodiments, the GUI objects associated with medical indicators on screen 200c of GUI 200 may be filtered to display a portion of the GUI objects. For example, as described above with respect to FIG. 2B, the user may choose to filter by status of the medical note for a medical consultation. For example, a user may select (e.g., tap/click) an icon on screen 200c and cause one or more filtering settings to be displayed for adding, removing, and/or modifying one or more GUI objects in screen 200c. In some embodiments, filtering by the status of the medical note may include whether the medical note is started (but not completed) and sent (but not yet signed). In some embodiments, the user may filter the GUI objects associated with medical consultations to display those which are set to expire in a customizable amount of time. For example, the user may customize the duration of time in which medical notes will be due (e.g., 1 day, 3 days, 1 week, 2 weeks, 1 month, etc.) to display the GUI objects associated with the medical consultations with medical notes that have a due date within the selected duration of time.

Upon selecting a GUI object corresponding to a medical consultation with a patient (e.g., on screen 200b illustrated in FIG. 2B), the user may be prompted to upload and/or begin an audio recording of the medical consultation. For example, the user may tap (e.g., click) on GUI object 210, and may be presented with a screen (not illustrated) comprising a button for beginning an audio recording. For example, screens 200n, 200o, and 200p illustrated with respect to FIGS. 2N, 20, and 2P, respectively, may comprise a selectable button (e.g., graphical object) 266 configured to start and/or stop an audio recording of a medical consultation. In some embodiments, the duration of the recording may be displayed on the screen. For example, the duration of the recording may be disposed adjacent to the recording button 266 on the screen (as shown in screens 200n, 200, and 200p). It is to be understood that button 266 may additionally or instead be overlaid on one or more of screens 200d-200m, albeit not explicitly illustrated.

Returning to FIG. 2C, in some embodiments, the user may select GUI object 208 corresponding to a medical consultation with a patient that already comprises medical information (e.g., from audio and/or transcript conversation data), and may add additional medical information (e.g., by adding additional transcript data and/or audio conversation data) to the existing medical consultation record. In some embodiments, the user may select GUI object 208 corresponding to a medical consultation that comprises audio and/or transcript conversation data, and may add, modify, and/or delete medical information (e.g., via dictation, typing, and/or click point GUI objects).

Once a medical consultation record comprises audio and/or transcript data, the user may visualize one or more screens on GUI 200 comprising different representations of the processed data. FIG. 2D depicts screen 200d of graphical user interface 200, in accordance with some embodiments. Screen 200d may depict GUI 200 upon the system completing processing of audio and/or transcript conversation data. For example, screen 200d may illustrate a transcript of the conversation in dialogue format. In some embodiments, the dialogue may be viewable real-time on screen 200d as it is being processed by the system (e.g., one or more components of system 102). In some embodiments, the dialogue may be viewed on screen 200d once the system completes the processing of the transcript conversation data.

GUI 200 may comprise a tab bar 216 comprising one or more selectable icons for toggling between screens of GUI 200. For example, screen 200d may be displayed upon selecting a dialogue icon 218. In some embodiments, the dialogue icon 218 may comprise one or more text and/or symbol portions (e.g., the word “visit,” a conversation bubble symbol, etc.) to indicate to the user that upon selecting the dialogue icon 218, the user may be presented with one or more dialogues corresponding to the medical consultation. In some embodiments, upon selecting (e.g., clicking/tapping) one or more icons on tab bar 216, the corresponding icon may be emphasized. For example, the icon may be visualized in a different color, bolded, outlined, highlighted in a colored shape, etc. from the remainder of the icons in tab bar 216.

In some embodiments, a medical consultation may comprise one or more inputs of audio conversation data and/or transcript data, and the user may toggle between inputs within the dialogue screen using a dialogue menu 220. For example, screen 200d may comprise a record of a medical consultation that comprises two inputs, as depicted by the two GUI objects in dialogue menu 220. The GUI objects in dialogue menu 220 may be associated with icons comprising information related to the inputs to differentiate multiple inputs from one another. For example, the GUI objects may be a symbol comprising a number, a time stamp corresponding to when the recording began and/or ended, the duration of the recording, etc. For example, as shown on screen 200d in FIG. 2D, a first input may be associated with an icon comprising the number “1” and the time at which the recording began (e.g., 12:27). A second input may be differentiated from the first input by the number “2” and the time at which the recording began (e.g., 1:19). In some embodiments, the icons for the multiple inputs may be displayed in a “carousel” such that the user may scroll (e.g., left/right or up/down) to toggle between inputs. In some embodiments, the icons of the multiple inputs may be buttons in dialogue menu 220. In some embodiments, the order in which the icons are displayed may be dependent on the time stamps and/or number associated with the input. For example, the second input in screen 200d may follow the first, at least because the time stamp occurs after the first input. A user may upload and/or record any number of inputs corresponding to the medical consultation and may toggle between the displayed dialogues of the inputs via dialogue menu 220 on screen 200d.

As mentioned above, the system may be configured to receive an input comprising audio conversation data, transcript conversation data, and/or existing electronic medical record (EMR) data. The system may process the audio conversation data to generate a transcript in dialogue format as displayed on screen 200d of GUI 200. For example, the system may process audio conversation data to generate transcript data using one or more automatic speech recognition (ASR) models/algorithms, associated with ASR engine 104. Based on the uploaded and/or generated transcript data, the system may be configured to process the data to determine portions of the transcript data corresponding to different parties speaking (e.g., medical professional, patient, etc.), however, in some embodiments, the transcript data may comprise only a single party speaking (e.g., the medical professional). The displayed dialogue may denote the different parties speaking in the transcript data, for example, using different colors/shadings and/or markers (e.g., “C” for clinician, “P” for patient, etc.). Screen 200d illustrates a dialogue between two parties comprising clinician (e.g., medical professional) dialogue portions 222 and patient dialogue portions 224. As shown, the different dialogue portions are distinguished using shading and markers. For example, the clinician portions 222 may not comprise shading, whereas the patient portions 224 may be shaded in color (e.g., grey). Thus, the user may easily distinguish between parties in the dialogue.

In addition to processing transcript data to identify and denote the parties speaking in the transcript data, the system may be configured to identify medical entities in the input data. For example, the system (e.g., system 102) may process the input transcript and/or EMR data using one or more natural language processing (NLP) models/algorithms (e.g., associated with NLP engine 106) to determine keywords, phrases, and/or sentences within the input data that may correspond to medical information of the patient. Example NLP models may include but are not limited to large language models (LLMs). The system may additionally be configured to summarize one or more portions of the transcript data, the summary insertable to the generated medical note. The identified medical entities may be highlighted within the displayed dialogue of the transcript data on screen 200d to indicate to the user what entities were automatically identified by the system. For example, the medical entity text may be highlighted with a block of color, bolded, italicized, or modified in comparison to the remainder of the dialogue text. In some embodiments, a combination of the techniques described may be applied to differentiate medical entities from the remainder of the dialogue. For example, medical entities 226 may be differentiated from the remainder of the dialogue using a different text color (e.g., the dialogue may be displayed in a standard black color, and the identified medical entities may be displayed in colored text, such as blue text). For example, the medical entities “back pain,” “metformin 500 mg,” and “diabetes” are emphasized in bold text in the dialogue displayed in screen 200d of GUI 200. By highlighting the medical entities identified by the system, the user may be able to efficiently identify key points of the medical consultation apart from the full dialogue. By identifying medical entities that have been automatically identified by the system, transparency and visibility may be provided to the user, such that the user is made aware of which entities have been gleaned from the application of one or more NLP models; this may help to facilitate the user's review and validation of the displayed information.

As mentioned above, the user may toggle between different screens of GUI 200 comprising information related to a medical consultation of a patient using one or more icons disposed in tab bar 216. FIG. 2E depicts a screen 200e of graphical user interface 200, in accordance with some embodiments. Screen 200e may depict a medical note outline window for the medical consultation. Screen 200e may display an outline of the medical consultation, the outline optionally divided based on complaints (e.g., problems) reported by the patient in the medical consultation. As described above, the system may determine one or more medical entities in the transcript data. The system may be configured to classify the medical entities, for example, as a complaint, symptom, treatment, test, etc. The medical entities classified as complaints may be inserted into a template displayed on screen 200e comprising an outline of the medical consultation. For example, the template (e.g., accessed from interface template library 112) may comprise a complaint menu 230, the complaint menu 230 comprising one or more GUI objects corresponding to the identified medical entities classified as complaints. Each of the complaints identified in the transcript data may be pre-populated (e.g., automatically inserted) into the complaint menu 230 and may be selectable (e.g., the user may click/tap) to toggle between displaying medical information related to the complaint on GUI 200. For example, the dialogue displayed in screen 200d of FIG. 2D identifies the medical entity “diabetes,” and the complaint menu 230 in screen 200e of FIG. 2E comprises a corresponding GUI object (e.g., button) labeled with the complaint “diabetes.”

In some embodiments, the number of complaints in a medical consultation may exceed the number of GUI objects viewable in complaint menu 230 at once, and complaint menu 230 may comprise a “carousel” such that the user may swipe left/right on screen 200e to view the complaints. In some embodiments, complaint menu 230 may comprise a drop-down menu displaying the one or more medical complaints determined from the transcript data of the medical consultation. For example, FIG. 2N of screen 200n illustrates a drop-down menu 230 of medical complaints, wherein each of the medical complaints may be a selectable graphical object. By selecting one or more of the medical complaints, a medical note outline corresponding to the selected complaint may be displayed (e.g., screens 200o and 200p illustrated with respect to FIGS. 20 and 2P, respectively). In some embodiments, the complaints displayed in complaint menu 230 may instead or additionally include one or more complaints previously reported by the patient in a past medical consultation (e.g., accessed from an electronic medical record stored in medical records library 118). As will be described in greater detail below with respect to FIG. 2F, in some embodiments, the user may add one or more complaints from a list of complaints to complaint menu 230. For example, the complaint menu 230 may comprise a selectable icon (e.g., a “+” sign) that may cause a list of potential complaints to be displayed to the user on GUI 200 for addition into the medical note outline.

In some embodiments, the window in screen 200e may comprise a plurality of medical indicators, including pre-populated indicators (e.g., by the system and/or from a template) and/or manually added indicators by a user in the medical note outline. The medical indicators may correspond to extracted medical entities from the transcript data (e.g., the dialogue displayed in the visit page of screen 200d) and suggested by the system, as well as one or more medical indicators suggested based on a template. The medical indicators may instead and/or additionally be suggested based on the patient's medical history (e.g., determined from information stored in medical records library 118), the clinician's specialty, etc. In some embodiments, the suggested medical indicators may be selected by the system from a library communicatively coupled to the system (e.g., medical record component library 114) and may be pre-populated in a template. The medical indicators may be organized in the medical note outline, the outline provided by a template (e.g., accessed from interface template library 112). For example, the outline may comprise different sections (e.g., regions) corresponding to sections of a standard medical note, such as history of present illness (HPI), review of systems (ROS), physical examination (PE), assessment/plan (A/P), etc.

FIG. 2E shows an HPI section 232 of a medical note outline. In some embodiments, the medical note outline may be displayed on screen 200e “accordion” style, such that the medical note sections may be toggled between being hidden and expanded. For example, a user may select (e.g., tap/click) an area on screen 200e proximate to the section label (e.g., HPI section 232) to toggle between an expanded version and a hidden version of the section. In some embodiments, the user may select a section of the medical note from a drop-down menu displayed on screen 200e to display the medical note outline corresponding to the selected section. In some embodiments, the medical note sections may be displayed in a grid comprising selectable icons corresponding with the medical note sections, and the user may select (e.g., tap/click) on an icon to cause the corresponding medical note section to be displayed on GUI 200. In some embodiments, the medical note outline may be scrollable, such that the user may click/tap and drag to view sections of the medical note outline one after the other. For example, the medical note outline for each complaint may be displayed one after the other as the user scrolls up/down and/or swipes left/right on the GUI 200.

FIG. 20 illustrates a screen 200o comprising hidden sections (e.g., hypertension, back pain, hyperlipidemia) and expanded sections (e.g., diabetes) of a medical note outline. As shown, an expanded section may comprise sub-sections that may additionally be toggled between a hidden state and an expanded state, as described in greater detail below. In some embodiments, upon selecting a complaint, a user may select a preset 268 for a medical note outline to be displayed for the selected complaint. For example, the medical note outline preset 268 may be selected based on the clinician preference, risk of the patient associated with the selected complaint, age of the patient, a combination of one or more of the aforementioned factors, etc.

Upon selecting a complaint in the medical note outline, a user may additionally or instead add a description 270 related to the selected complaint. For example, the description may be related to one or more sub-sections, or may be related to none of the listed sub-sections. In some embodiments, the clinician may add, edit, and/or remove patient information in description 270 by typing text, recording audio, and/or selecting one or more words/phrases from a set of predetermined words/phrases.

Each of the sections of the medical note may be organized into an outline on GUI 200 based on one or more sub-sections. For example, as shown in FIG. 2E, the HPI section 232 may be organized into sub-sections including medication sub-section 234, symptoms sub-section 236, treatments sub-section 238, and tests sub-section 240. FIG. 20 may show an additional assessment sub-section 272. For example, medication sub-section 234 may comprise information related to medications the patient has previously and/or is currently taking related to the complaint. Symptoms sub-section 236 may comprise medical information related to symptoms the patient is currently experiencing and/or has experienced in the past related to the complaint. Treatments sub-section 238 may comprise medical information related to treatments the patient has previously attempted and/or been prescribed related to the complaint. Tests sub-section 240 may comprise medical information related to tests the patient has previously been prescribed related to the complaint. Assessment sub-section 272 may comprise medical information related to the clinician's assessment of the patient in relation to the selected complaint.

In some embodiments, HPI section 232 may comprise additional sub-sections not illustrated in screen 200e of FIG. 2E. For example, HPI section 232 may include sub-sections such as timing of the complaint, description of the complaint, anatomical location of the complaint, and history of the complaint. In some embodiments, the other sections of the medical note (e.g., ROS, PE, A/P, etc. not illustrated in FIG. 2E) may be outlined in the medical note outline using one or more similar and/or different sub-sections. For example, an assessment/plan section may comprise sub-sections including assessment of the complaint, medication to be described for the complaint, treatment for the complaint, and tests to be ordered for the complaint. The sections and/or sub-sections displayed in the outline on screen 200e may be based on a template (e.g., from interface template library 112) and/or data from a medical record component library 114. In some embodiments, the medical note outline may not comprise a section and sub-section outline, and rather may combine one or more of the above-mentioned sub-sections into a single section associated with a given complaint (e.g., as illustrated at least in FIGS. 20-2P).

The system may be configured to organize the medical indicators mentioned above (e.g., extracted as medical entities from transcript data and/or suggested based on one or more factors, such as the extracted medical entities, patient medical history, clinician specialty, etc.) based on the complaint the medical indicator corresponds to, as well as the section and/or sub-section the medical indicator corresponds to. For example, as mentioned above, the system may be configured to classify an extracted medical entity. In classifying the medical entity, the system may identify if the entity is a complaint, and if not, rather what complaint the medical entity is related to. The system may then classify the medical entity based on section (e.g., HPI, PE, ROS, A/P, etc.), and/or sub-section (e.g., medications, symptoms, treatments, tests, etc.) within the corresponding complaint that the medical entity is related to. The medical entity may be represented in the medical outline on screen 200e as a medical indicator, in that a medical indicator differs from a medical entity at least because it comprises one or more interactive features (e.g., the medical indicator is a GUI object the user may select to cause the GUI object to change). For example, as described in greater detail below, the user may select a medical indicator to change the state of the indicator, which may influence the medical note composed by the system.

With reference to FIG. 2E, the system may be configured to identify medical entities as symptoms of the complaint “diabetes,” such as “change in vision,” “chest pain,” “polyuria,” “fatigue,” “weakness,” “dizziness,” “numbness/tingling,” and “foot pain/sores,” and may display the symptoms as medical indicators within the symptom sub-section 236. In some embodiments, one or more of the symptoms may be extracted from the transcript data (e.g., dialogue displayed on screen 200d of FIG. 2D). In some embodiments, one or more of the symptoms may be pre-populated in a template, for example, based on known symptoms associated with the complaint (e.g., stored in medical record component library 114). In some embodiments, one or more of the symptoms may be pre-populated based on patient medical history and/or clinician specialty. As will be described in greater detail below with regards to FIG. 2G and the medication sub-section 234 of the medical note outline, the user may be able to add and/or remove one or more medical indicators from the displayed medical note outline.

In some embodiments, medical note sub-sections such as treatment sub-section 238 and/or test sub-section 240 may comprise one or more suggested (e.g., pre-populated) medical entities, represented as medical indicators in the medical note outline as described above with respect to symptoms sub-section 236. For example, treatment sub-section 238 may comprise medical indicators such as “following diabetic diet” and “getting exercise.” Test sub-section may comprise medical indicators such as “HbgA1c,” “morning glucose range,” “mid-day glucose range,” and “evening glucose range.” As mentioned above with respect to symptoms sub-section 236 and will be described in greater detail below with respect to FIG. 2G, the user may add, modify, and/or remove one or more medical indicators from the displayed medical note outline.

As mentioned above, medical indicators may differ from medical entities in that the medical indicators may be associated with a modifiable status. Medical indicators may be suggested as a component of a template (e.g., based on the complaint type), suggested by the system (e.g., based on extracted medical entities) and/or manually added by a user (e.g., by typing the medical entity, selecting from a list, etc.). In some embodiments, each of the suggested medical indicators may be viewable as present or absent in the medical consultation. For example, a template may comprise suggested present and/or suggested absent medical indicators, as well as the system may suggest present and/or absent medical indicators. The user may add and/or remove additional medical indicators and may modify existing medical indicators. Modifying medical indicators, as will be described in greater detail below, may comprise changing the state of the medical indicator. For example, the state of medical indicators may be altered between one or more of “suggested present,” “suggested absent,” “confirmed present,” and/or “confirmed absent.”

As shown in FIG. 2E, the medical indicators in the symptoms sub-section 236 are illustrated in an absent state, such as a “suggested absent” state 246. In some embodiments, the “suggested absent” state 246 may be associated with strikethrough text. For example, the text describing the medical indicator may be crossed out, as shown in symptoms sub-section 236 of screen 200e. In some embodiments, the “suggested absent” state may additionally or instead be represented by a different color of text, such as a light-colored (e.g., semi-transparent) text. A medical indicator may be denoted as “suggested absent” when the system extracts the corresponding medical entity from the transcript data and the medical entity is associated with one or more words/phrases that allow the system to conclude that the medical indicator corresponding to the entity is absent (e.g., not present). For example, a portion of the dialogue may show that the medical professional (e.g., clinician) has stated “Have you experienced any numbness or tingling in the hands and feet?” in relation to the complaint “diabetes,” and in response, the patient has stated “Nope. It's all fine.” The system may be configured to process the transcript data to determine that the patient is not experiencing the symptom of numbness/tingling in the hands and feet and may suggest the absence of this symptom accordingly in the medical note outline within the symptom sub-section 236 of the HPI section 232 corresponding to the complaint “diabetes” (e.g., by striking through the symptom “numbness/tingling”).

In some embodiments, a “suggested absent” medical indicator, such as medical indicator 246, may be suggested absent as part of a template. For example, the system may suggest one or more medical indicators that may be absent from the medical consultation based on a template (e.g., corresponding to a complaint type), patient medical history, clinician specialty, etc. In some embodiments, the system may be configured to receive analytics from the front-end system (e.g., front-end user system 108 described above with respect to FIG. 1) regarding usage trends of GUI 200, and the system may be configured to display (or remove from display) suggested present and/or absent medical indicators based on the trends.

In addition to a “suggested absent” medical indicator, as mentioned above, the status of one or more medical indicators may be “suggested present.” For example, the system may be configured to extract medical entities and may display suggested medical indicators related to the extracted medical entities in one or more sub-sections of the medical note outline. In some embodiments, at least a portion of the “suggested present” medical indicators may not be explicitly stated as a medical entity within the transcript data. Thus, the system may be configured to infer one or more present medical indicators based on medical entities in the transcript data. In some embodiments, similar to the “suggested absent” medical indicators described above, “suggested present” medical indicators may be suggested based on a template (e.g., corresponding to the complaint type), trends, patient medical history, clinician specialty, etc. For example, the system may determine that an extracted medical entity is a complaint and may be configured to pre-populate a medical note outline template based on the complaint type.

With reference to FIG. 2E and screen 200e, the medical indicators within treatment sub-section 238 may illustrate medical indicators in a “suggested present” state 244 associated with the complaint “diabetes,” wherein these medical indicators may be suggested (e.g., pre-populated) based on a template related to the complaint. The “suggested present” medical indicators that are pre-populated based on a template, patient medical history, clinician specialty, trends, etc. may be differentiated from other medical indicators (e.g., those suggested based on direct extraction from transcript data, described below) using italicized and/or different colored text (e.g., light-colored, such as semi-transparent text). As will be described in greater detail below, at least a portion of the “suggested present” and “suggested absent” medical indicators, such as those pre-populated based on a template, may otherwise be referred to as “inactive” in that the user may be required to modify the status of the medical indicators to cause the indicator to be included in a later generated output (e.g., medical record).

As mentioned above, the “suggested present” medical indicators may be suggested based on the medical entities extracted from the input (e.g., transcript) data. In some embodiments, a medical indicator may instead or additionally be denoted as “suggested present” when the system extracts the medical entity from the transcript data and the medical entity is associated with one or more words/phrases that allow the system to conclude that the medical indicator corresponding to the entity is present. For example, a portion of the dialogue may show that the medical professional (e.g., clinician) has stated “You're taking metformin 500 mg 1 tab twice a day, for your diabetes?” and in response, the patient has stated “Yes.” The system may be configured to process the transcript data to determine that the patient is taking 500 mg of metformin (1 tab) twice a day and may indicate this medication accordingly in the medical note outline within the medication sub-section 234 of the HPI section 232 corresponding to the complaint “diabetes” (not illustrated, at least because medication sub-section 234 may be in a collapsed view). The medical indicators in the test sub-section may illustrate a “suggested present” state 242 based on medical entities extracted from the transcript data. For example, an HbgA1c level and morning glucose range may be extracted from the transcript data and illustrated in the medical note outline as a “suggested present” medical indicator.

In another example, the medical indicator acetaminophen and the related dosage information on screen 200p of FIG. 2P may illustrate a “suggested present” state 242. For example, in comparison at least to the naproxen medical indicator, the text of the medical indicator in the “suggested present” state 242 may be emphasized (e.g., underlined, highlighted, bolded, italicized, colored, etc.) to differentiate the state of the different medical indicators. In some embodiments, the naproxen medical indicator may be in a “confirmed present” state 276, at least because the patient information associated with the medical indicator may have been confirmed by the clinician. The “confirmed present” and “confirmed absent” states are described in greater detail below.

The “suggested present” medical indicators corresponding directly to medical entities extracted from the transcript data may differ from those suggested based on a template, for example, in that those directly extracted from transcript data may not require confirmation from a user (e.g., the state may not be required to be changed) for the medical indicator to be included in a later generated output. In some embodiments, the different types of “suggested present” medical indicators (or “suggested absent” medical indicators, for that matter) may be differentiated from each other. For example, “suggested present” and/or “suggested absent” medical indicators directly corresponding to medical entities extracted from transcript data may be highlighted in a different style (e.g., bold text) and/or color of text (e.g., blue text). On the other hand, those that are suggested based on a template, patient medical history, clinician specialty, trends, and/or are indirectly related to an extracted medical entity may be illustrated in light-colored (e.g., transparent) text and/or italicized.

In some embodiments, “suggested present” medical indicators extracted from transcript data may have text in a first color (e.g., blue), and the corresponding medical entities may be colored in the same color in the transcript data (e.g., dialogue) on screen 200d. Thus, a user may toggle between screens (e.g., via tab bar 216) and identify corresponding medical entities in the dialogue of screen 200d for at least a portion of the “suggested present” medical indicators in screen 200e. In some embodiments, at least a portion of the medical entities corresponding to medical indicators that are “suggested absent” based on medical entities extracted from the transcript data may additionally be demarcated in the dialogue of 200d, for example using a different emphasis technique from the medical entities corresponding to “suggested present” (e.g., different text color, style, etc.).

As mentioned above, a “suggested” (e.g., “suggested present” and/or “suggested absent”) medical indicator may be toggled between different states. For example, a user may select (e.g., tap/click) a medical indicator to modify the state of the indicator. In some embodiments, by tapping/clicking a medical indicator one or more times, the user may activate and toggle the state of the medical indicator from “suggested present” and “suggested absent” to “confirmed present” and/or “confirmed absent.” For example, the system may automatically classify a medical indicator as “suggested absent” (e.g., based on a template), and the user may tap/click the medical indicator (e.g., once) to change the status to “confirmed absent.” In some embodiments, the user may tap/click the medical indicator again (e.g., a second time) to change the status to “confirmed present.” The user may toggle between the states an indefinite number of times. In some embodiments, the user may tap/click and hold a medical indicator for a pre-defined amount of time to change the status. For example, the user may tap/click the medical indicator and hold to be presented with a drop-down menu of status options (not illustrated). In some embodiments, the user may also have the option to delete the medical indicator from the outline on screen 200e. For example, by tapping/clicking and holding, the user may additionally or instead be presented with the option to delete the medical indicator.

In some embodiments, the user may choose to not click/tap on a “suggested” medical indicator in the medical note outline. By not activating these medical indicators, the system may determine that the medical indicators should not be included in the composition of the medical note, whereas those that are “confirmed present” and “confirmed absent” may be included in the medical note, as will be described in greater detail below at least with respect to FIG. 2J. In some embodiments, the user may be required to activate all “suggested” medical indicators to either a “confirmed present” or “confirmed absent” state, or the user may delete irrelevant “suggested” medical indicators from the medical note outline in GUI 200 prior to completion of the medical note. In some embodiments, as mentioned above, at least the medical indicators suggested based on medical entities extracted from transcript data may not require user confirmation (e.g., changing state from suggested to confirmed) to be included in the output medical record. On the other hand, medical indicators suggested based on a template, patient medical history, clinician specialty, data trends, etc. may require user validation to be included in a later generated output. The “confirmed” medical indicators may be differentiated from at least a portion of those with a status “suggested” (e.g., “suggested present” and/or “suggested absent”) for example, by highlighting the text of the medical indicator and/or changing the color of the text in comparison to the remainder of the text in the outline.

In some embodiments, medical indicators may be associated with one or more text fields. For example, the medical indicators within the test sub-section 240 may comprise text fields, wherein a number (e.g., value) is associated with a given test. In some embodiments, the text field may automatically be pre-populated by the system, for example, by extracting a corresponding medical entity from the transcript data. In some embodiments, the system may extract the type of test, but may require a user to input an associated value with the test (or vice versa). In some embodiments, the user may modify the value associated with a displayed test, for example, by tapping/clicking the text field. Upon selecting the text field (e.g., empty or prefilled with an extracted value), the user may be presented with a numerical keypad (e.g., to freely type in a value), a picker comprising a list of pre-defined selectable values, a stepper for incrementing a value in a pre-defined manner, etc. (not illustrated). In some embodiments, the selectable values may be based on the corresponding test (e.g., stored in medical record component library 114). In some embodiments, the user may toggle between inputting values in any way described above (e.g., based on a template stored in interface template library 112).

As mentioned above, in some embodiments, the user may be required to complete one or more sections and/or sub-sections of a medical note prior to sending the medical record (e.g., medical note) to an electronic health records library. For example, the system may notify (e.g., alert) the user prior to attempting to generate a medical record based on the medical note outline and/or prior to the user attempting to send the medical note to the library that one or more sections and/or sub-sections of the medical note are incomplete. In some embodiments, a sub-section may comprise one or more medical indicators that require user confirmation. For example, with reference to FIG. 2E, the clinician (e.g., user) may have inquired regarding the patient's morning glucose range during the medical consultation, which was captured by the system and is displayed in a medical indicator on screen 200e in FIG. 2E. However, the clinician may have failed to inquire regarding a mid-day and/or evening glucose range of the patient (illustrated as empty text fields on screen 200e in FIG. 2E). The system may notify the user to complete these fields prior to attempting to send the medical note. In some embodiments, GUI 200 may be used by the medical professional as the medical consultation progresses, thus the system may remind the user via GUI 200 to request the required medical information prior to completion of the consultation to complete one or more medical indicators.

In some embodiments, the system may be configured to generate a medical note despite a lack of medical indicators in one or more sections and/or sub-sections of the medical note outline. In some embodiments, the system may require user confirmation that a medical note section and/or sub-section is complete, at least in the instance the section/sub-section does not comprise any medical indicators.

As mentioned above, a user may manually add one or more complaints to a complaint menu of the medical note outline, and the user may also add one or more medical indicators to a section and/or sub-section of the medical note outline. For example, the user may tap/click on a selectable icon (e.g., a “+” symbol, a text label, etc.) on screen 200e (e.g., associated with complaint menu 230) to cause a selection of potential complaints to be displayed on GUI 200. FIG. 2F depicts a screen 200f of graphical user interface 200, in accordance with some embodiments. Screen 200f may display a complaint (e.g., problem) selection 248 that may be added to the medical note outline.

In some embodiments, the selection of complaint options 248 may be organized (e.g., filtered), for example, at least by active and common complaints. For example, the screen 200f may comprise a GUI object (e.g., button) for each of the active and common complaint categories, and the user may select the corresponding GUI objects to view the sorted selection of complaints. Active complaints may be defined as complaints identified within the patient medical history and/or in the transcript data corresponding to the current medical consultation. Common complaints may be defined complaints commonly identified (e.g., based on usage trends of the clinician, the specialty of the clinician, common trends in healthcare, etc.). One or both selections of potential complaints may be dynamically updated by the system based on usage data and/or data stored in medical records library 118, for example. In some embodiments, the user may be able to search one or both selections simultaneously for a medical complaint of interest to add to the medical note outline.

In some embodiments, the complaint options may be provided in a scrollable list with checkboxes. For example, as shown in FIG. 2F, complaints previously identified by the system and/or manually selected by the user may be associated with an activated checkbox (e.g., a check is visible), whereas those which are not currently selected may be associated with a deactivated checkbox (e.g., the checkbox is empty). The user may tap/click on GUI 200 proximate to a given complaint to select/deselect the complaint. For example, the user may tap on the checkbox, the text describing the complaint, or another area proximate to the icon and/or text field to select/deselect the complaint. By selecting one or more complaints from the selection of complaint options 248, the selected complaints may be displayed in a complaint menu of the medical note outline (e.g., complaint menu 230 on screen 200e in FIG. 2E).

In some embodiments, the complaint options may be associated with a classification based on visit type. Visit type may include, for example, chronic, acute, and wellness. For example, as shown in FIG. 2F, “diabetes” may be classified as chronic, “annual wellness” may be classified as wellness, and “back pain” may be classified as acute. The complaint options may be associated with another classification, such as medical specialty, anatomical location of the complaint, etc. and this classification may additionally or instead be displayed in the complaint selection 248. In some embodiments, the user may filter the selection of complaints 248, for example, based on the classification(s), active complaints, and common complaints (as mentioned above). In some embodiments, one or more complaints may be classified as both a common and an active complaint, and thus may appear in each of the categories upon filtering the problems. In some embodiments, the complaints may be sorted (e.g., by relevance, alphabetical (A-Z), alphabetical (Z-A), popularity, etc.).

In some embodiments, the potential complaints displayed in complaint list 248 may be displayed in another format different from a list with checkboxes, as described above. For example, an interface template (e.g., from interface template library 112) may be applied that displays the complaint options as icons in a grid, a carousel (e.g., a limited number of options are viewable at one time and the user may swipe left/right to view options), and/or a drop-down menu. In some embodiments, rather than listing each of the complaint options with a selectable checkbox, the complaints may be selected/deselected by tapping/clicking the text and/or an area on GUI 200 proximate to the text describing the complaint, which may cause the complaint to be highlighted (e.g., the text may be outlined, the style and/or color of the text may change, etc.).

As mentioned above, a user may add one or medical indicators to a medical note outline section and/or sub-section. FIG. 2G depicts a screen 200g of graphical user interface 200, in accordance with some embodiments. Screen 200g may display a selection of medical entity options that may be added to the medical note outline. For example, screen 200g may display a selection of medications 250 when a user selects a corresponding icon (e.g., as described above, a label stating “add,” a “+” symbol, etc.) proximate to the medication sub-section in the medical note outline. In some embodiments, the selection of medications 250 may be displayed in a list, as shown on screen 200g. In some embodiments, as described above with the complaint selection, the medical entity options (e.g., medications) may be provided in a different format (e.g., grid with icons, carousel, list with checkboxes, etc.). In some embodiments, a user may add medical entities to any of the sections and/or sub-sections described above in a similar manner as that which is described with respect to the medication section. For example, the symptoms sub-section, treatments sub-section, and tests sub-section may comprise a similar selectable icon that the user may click/tap to cause a selection of corresponding medical entities to be displayed on the screen for addition into the medical note outline. The medical entity options may be provided within the corresponding sections/sub-sections as medical indicators, such that the indicator may be selectable to change the state of the indicator as needed (e.g., from “confirmed present” to “confirmed absent,” or vice versa).

In some embodiments, the selection of medications 250 may be customized such that the provided medications correspond to the complaint (e.g., problem) that the user is currently assessing. For example, if the complaint is “diabetes” as shown on screen 200f, the list of medications may be those which are known to be associated with diabetes. The system may be configured to reference a library of medications associated with a given complaint (e.g., medical record component library 114) to determine which medications to display in medication selection 250 on GUI 200. In some embodiments, the selection of medications 250 may be based on the clinician's specialty, patient's medical history, the clinician's trends, etc. In some embodiments, the selection of medications 250 may be based on a combination of the above. The selection of medications may be sorted in a list, for example, in alphabetical order (e.g., A-Z or Z-A), by relevance, and/or by popularity. In some embodiments, the user may be able to search and/or manually add a medication by selecting a search bar associated with the medication selection 250, which may cause a keyboard to be displayed. In some embodiments, the user may dictate (e.g., speak) the medication of interest rather than manually typing into the keyboard to add the medication to the medical outline.

Each of the medications in the selection of medications 250 may be associated with selectable GUI objects, such that the user may click/tap the text describing the medication and/or an area proximate to the text on screen 200g to select the medication and cause it to be displayed as a medical indicator within the medication sub-section of the medical note.

In some embodiments, by selecting medication in the selection of medications 250 on screen 200g, a selection of dosages for the medication may be displayed on GUI 200. FIG. 2H depicts a screen 200h of graphical user interface 200, in accordance with some embodiments. Screen 200h may display a selection of potential dosages 252 (and in some embodiments, frequencies associated with the dosages) for a given medication (e.g., selected from the selection of medications 250 in FIG. 2G). In some embodiments, the selection of potential dosages may correspond with the medication selected, such that standard dosages of the medication are displayed (e.g., based on data stored in medical record component library 114). For example, as shown in FIG. 2H, for the medication “Pravastatin,” the potential dosages may be displayed in a list, including “15 mg, QD” (e.g., 15 mg once a day), “100 mg, TID” (e.g., 100 mg three times a day), “200 mg, QD,” “400 mg, QD,” and/or “500 mg, QD.” In some embodiments, the user may manually input a dosage of the medication using an input field (e.g., search field). For example, the user may click/tap a search field (e.g., search bar) to cause a keypad comprising at least numbers and units to be displayed for the user to input a dosage of the medication and/or frequency associated with the dosage. In some embodiments, upon selecting a medication to be added to the medical note outline, the user may be presented with a dropdown menu, grid with icons, carousel, picker, and/or stepper (e.g., the user may modify the dosage by increasing/decreasing by a predetermined value between numbers) for selecting a dosage associated with the medication. In some embodiments, the user may be presented with a first type of selection tool (e.g., dropdown menu, grid with icons, carousel, etc. described above) to select a dosage, and a second type of selection tool to select a frequency of dosage (e.g., QD, TID, etc.). The medication and corresponding dosage and/or dosage frequency may be displayed as a medical indicator within the medication sub-section of the medical note outline.

In some embodiments, upon clicking/tapping a medical indicator, the user may be presented with a predetermined set of options (e.g., modifiers) corresponding to the medical indicator that refine the indicator. FIG. 21 depicts a screen 200i of graphical user interface 200, in accordance with some embodiments. Screen 200i may display a selection of modifiers 254 associated with a medical indicator, such that the modifiers further refine the medical indicator. For example, the selection of modifiers 254 may be displayed in a dropdown menu, such that the user may click/tap on a modifier in the dropdown menu to add it to the medical note outline associated with the medical indicator. For example, screen 200i on FIG. 21 may depict a selection of modifiers 254 displayed as a dropdown menu and associated with the medical indicator “progression” within the assessment sub-section of the assessment/plan section of the medical note outline. The medical indicator “progression” may be associated with a predetermined set of modifiers, for example, “controlled,” and “uncontrolled.” In some embodiments, the user may toggle between modifiers by selecting the corresponding medical indicator on GUI 200 to toggle between potential modifiers. For example, the user may select (e.g., click/tap) the medical indicator a first time to display the modifier “controlled” associated with “progression,” and by clicking a second time, the user may cause GUI 200 to display the modifier “uncontrolled” in place of “controlled.” In some embodiments, the modifiers of a given medical indicator may be stored in a library (e.g., medical record component library 114). For example, the modifiers related to “progression” may be stored associated with “progression,” which may be distinct from those associated with “status” (e.g., displayed on screen 200i within the assessment sub-section in addition to “progression”).

As mentioned above, GUI 200 may comprise a plurality of windows (e.g., screens) that the user may toggle between while composing a medical note of a medical consultation with a patient. For example, the user may toggle between a dialogue window (e.g., illustrated in FIG. 2D), a medical note outline window (e.g., illustrated in FIG. 2E), and, as described in greater detail below, a medical note window. FIG. 2J depicts a screen 200j of graphical user interface 200, in accordance with some embodiments. Screen 200j may display a completed and/or partially completed medical note for a medical consultation with a patient. For example, at any time during use of GUI 200, the user may toggle between windows (e.g., via tab bar 216) to view the dialogue of the medical note, an outline of the medical note, and a composed medical note comprising automatically generated statements (e.g., sentences, phrases, or other portions of the medical note). By clicking/tapping on a medical note icon 256 within tab bar 216, the user may be presented with the medical note on screen 200j.

In some embodiments, the system may be configured to generate a medical note (and/or phrases or sentences for inclusion in a note) using at least the medical indicators comprised in the medical note outline. For example, the system may comprise templates (e.g., stored in output template library 116) for portions (e.g., phrases, sentences, etc.) of medical notes in which the system is configured to input the medical indicators from the outline into to generate complete sentences and/or phrases in a standardized format for inserting into the medical note. As mentioned above, the system may additionally be configured to generate other types of medical records, such as medical coding, prescription and/or lab test orders, after-care summaries, pre-visit charting, etc. Moreover, the system may be configured to summarize one or more portions of the input transcript data and may include the summary in the generated medical note.

For example, for a medical note, the symptom medical indicators 246 in screen 200e of FIG. 2E (e.g., “change of vision,” “chest pain,” “polyuria,” “fatigue,” “weakness,” “dizziness,” “numbness/tingling,” “foot pain/sores,” etc.) may be inserted into one or more statements within the symptoms sub-section of the medical note, as shown in the statement “He denies associated polyuria, polydipsia, change in vision, foot pain/sores, chest pain, weakness, fatigue, dizziness, and numbness and tingling,” on screen 200j in FIG. 2J. The medical indicators may be inserted into a statement corresponding with the state of the medical indicator. For example, the medical indicators associated with the example provided statement may be illustrated in a “confirmed absent” state, thus the statement with the symptom medical indicators begins with “He denies” to express the absence of the symptoms. In some embodiments, each medical indicator in the outline may be inserted into a single statement (e.g., each statement corresponds with one medical indicator). In some embodiments, multiple medical indicators may be aggregated into a single statement and/or phrase, as shown in the example described above.

In some embodiments, phrases and/or sentences generated based on medical indicators may instead or additionally be displayed in the medical note outline. For example, FIG. 2P illustrates generated phrases 274 “The patient denies any other symptoms,” and “His BP in office today is within normal limits” in each of the symptoms and assessment sub-sections, respectively. In some embodiments, one or more of the sentences and/or phrases 274 may be manually inputted by a user (e.g., by typing, recording, or selecting words/phrases).

The templates stored in output template library 116 may comprise a paragraph template, document template, and/or section template for a medical record, such as a medical note. As mentioned above, the templates of the medical record (and/or for portions thereof) may be generated (e.g., by a back-end user of back-end user system 110) based on the healthcare system, clinician's specialty, etc. As shown in screen 200j of FIG. 2J, the structure of the medical note (e.g., headers) may correspond to the structure of the medical note outline. For example, the medical note may comprise a section label corresponding to a section (e.g., HPI, PE, ROS, A/P, etc.), and the medical information may be organized within the section into sub-sections grouped by complaint. For example, in screen 200j, the section label HPI may comprise a text header of the complaint “diabetes,” followed by medical information grouped according to the sub-sections “medication,” “symptoms,” “treatment,” and “tests.” Following the medical information related to “diabetes,” the section HPI may comprise medical information for additional complaints. For example, screen 200j may comprise the text header “hypertension” following the medical information related to “diabetes.” The user may scroll on screen 200j (e.g., by clicking/touching GUI 200 and dragging) to view additional medical information from the medical consultation. In some embodiments, the medical note may be organized “accordion” style, such that the user may select one or more headers within the medical note (e.g., section labels, sub-section labels, and/or complaint/problem text headers) to expand/collapse a corresponding portion of the medical note.

In some embodiments, the user may add medical information related to the medical consultation with the patient directly into the medical note automatically generated by the system. FIGS. 2K-2M depict screens 200k, 2001, and 200m, respectively, in accordance with some embodiments. Screen 200k may display a menu 258 with icons, each icon corresponding to a method that the user may add additional medical information to the medical note. For example, the user may navigate to an area of the medical note that they desire to add medical information to, and may select the GUI (e.g., by clicking/tapping) to cause menu 258 to be displayed with options for adding medical information to the note at a cursor location corresponding with the area selected on the GUI. For example, the user may select (e.g., by clicking/tapping) the dictation button 260 to dictate (e.g., voice record) medical information into the medical note at a selected location. The dictation button 260 may be associated with an icon representative of dictation (e.g., a microphone). In some embodiments, the user may tap and hold a GUI object on screen 200k (e.g., button 260 or another individual GUI object, for example, a recording button) to capture medical information as dictation related to the medical consultation.

In some embodiments, as the user is dictating the medical information, the system may be configured to automatically process the audio data, generate transcript data, and display the transcript data in the medical note on screen 200k in real-time as it is being dictated by the user. For example, the user may speak “Related complications include high blood pressure, nephropathy manifested as proteinuria,” and the system may be configured to reproduce the medical information as text on screen 200k.

In some embodiments, in addition to or instead of dictating medical information into the medical note, the user may manually type the medical information. Screen 2001 of FIG. 2L may display a keyboard for manually inputting medical information to the medical note. The user may select the keyboard GUI object 262 in menu 258 to cause the keyboard to be displayed on screen 2001 of GUI 200, and the typed text may be inserted at a desired location clicked/tapped on the GUI (e.g., corresponding to the location of a cursor).

In some embodiments, in addition to or instead of dictating and/or manually typing medical information into the medical note, the user may select from a plurality of prefilled medical phrases to add medical information into the medical note. Screen 200m of FIG. 2M may display a selection of medical phrases that may be selectable and associated with one or more stored statements. For example, the user may select medical phrase button 264 in menu 258 to cause a selection of medical phrases to be displayed. The user may select (e.g., click/tap) one or more medical phrases, and the system may be configured to generate and display a statement and/or phrase in the medical note corresponding to the selected medical phrase. In some embodiments, the selection of medical phrases may be prefilled based on one or more factors, such as the associated complaint. For example, if the cursor is placed within the medical note in an area associated with the complaint “back pain,” the displayed medical phrases may be those related to back pain (e.g., as stored in medical record component library 114). In some embodiments, the selection of medical phrases may be displayed in a list on screen 200m that may be based on the clinician's specialty, clinician's usage trends, patient's medical history, etc. In some embodiments, the selection of displayed medical phrases may be based on a combination of the above. In some embodiments, the selection of medical phrases may be displayed in any manner as described above with respect to the selection of medications and/or selection of complaints. In some embodiments, the user may be able to search for a medical phrase (e.g., by clicking/tapping a search field associated with the list of medical phrases). The selection of medical phrases may be sorted in a list, for example, by relevance, popularity, alphabetical order (e.g., A-Z or Z-A), etc.

Upon completing the medical note, the user may review the medical note and send the medical note to an electronic health record (EHR) of the patient (e.g., medical records library 118). In some embodiments, the system may require the user to sign the medical note (e.g., electronically) to verify that it is complete before and/or after the medical note is sent to the EHR.

Method for Generating and Displaying Medical Records

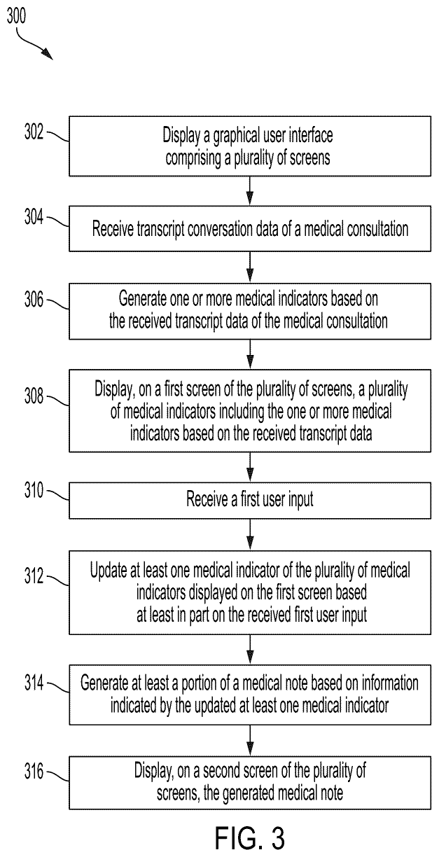

FIG. 3 depicts a flowchart describing a method 300 for generating and displaying a medical note based on audio and/or transcript conversation data. In some embodiments, method 300 may be performed by a system for providing a medical record generation platform, such as system 100 described above with reference to FIG. 1.

At block 302, the system may display a graphical user interface (GUI) comprising a plurality of screens. In some embodiments, the GUI may be embodied in a mobile user interface additionally comprising one or more touch-sensitive screens. In some embodiments, the GUI may be displayed on a desktop, workstation, personal computer (PC), and/or mobile device.

At block 304, the system may receive transcript conversation data of a medical consultation. In some embodiments, the system may initially receive audio conversation data and may process the audio data to generate transcript data for further processing. In some embodiments, the audio and/or transcript conversation data may comprise multi-party conversation data (e.g., between a patient and medical professional). In some embodiments, the audio and/or transcript conversation data may be dictation of a single party (e.g., a medical professional).

At block 306, the system may generate one or more medical indicators based on the received transcript data. In some embodiments, generating the medical indicators may comprise determining (e.g., identifying, extracting, etc.) medical entities from the received transcript data. For example, audio data may be processed (e.g., using one or more automatic speech recognition models) to generate transcript data. The transcript data may be processed using one or more natural language processing models to determine medical entities that correspond to medical indicators. In some embodiments, medical indicators may differ from medical entities in that medical entities may refer to the term, phrase, sentence, etc. extracted from the transcript data, whereas the medical indicator may correspond with (e.g., comprise) the medical entity and may be an object (e.g., icon, label) on the graphical user interface that a user may engage with (e.g., click, tap).

At block 308, the system may display on a first screen a plurality of medical indicators including the one or more medical indicators based on the received transcript data. In some embodiments, one or more displayed medical indicators may be pre-populated on the first screen based on a complaint in the transcript conversation data determined by the system. In some embodiments, the plurality of medical indicators may correspond to medical entities extracted by the system from the transcript conversation data.

At block 310, the system may receive a first user input. In some embodiments, the user input may comprise medical information that may modify one or more medical indicators. In some embodiments, the user input may cause addition of one or more medical indicators not previously displayed on the first screen.

At block 312, the system may update at least one medical indicator of the plurality of medical indicators displayed on the first screen based at least in part on the received user input. In some embodiments, updating the medical indicator may comprise changing the state of the medical indicator (e.g., from “suggested present” to “suggested absent,” “confirmed present,” and/or “confirmed absent,” etc.). In some embodiments, updating the medical indicator may comprise removing the medical indicator from the first screen. In some embodiments, updating the medical indicator may comprise adding a medical indicator based on a selection from a user.

At block 314, the system may generate at least a portion of a medical note based on information indicated by the updated medical indicator. In some embodiments, a generated medical note portion may comprise one or more statements comprising a plurality of medical indicators. In some embodiments, each medical indicator may correspond to a single generated statement in the medical note.

At block 316, the system may display, on a second screen of the plurality of screens, the generated medical note. In some embodiments, the medical note may comprise a plurality of automatically generated statements. In some embodiments, the structure of the statement(s) and/or medical note may be based on a template (e.g., stored in output template library 116). In some embodiments, the system may be configured to receive additional user inputs at the second screen comprising medical information for insertion to the medical note.

### Device for Generating and Displaying Medical Records

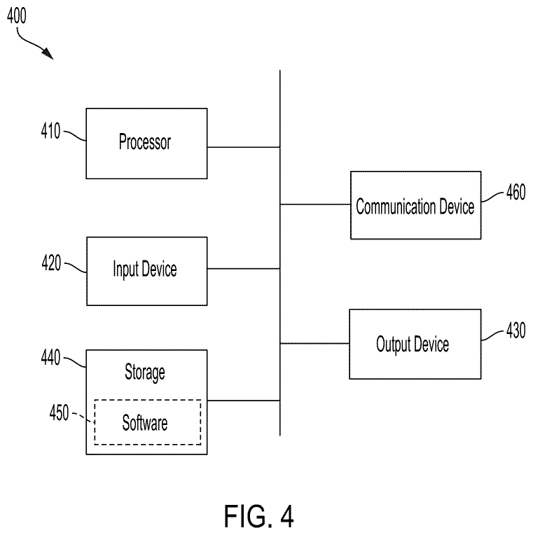

FIG. 4 illustrates an example of a computer, according to some embodiments. In some embodiments, computer 400 may execute a method for automatically generating and displaying medical notes.

Computer 400 can be a host computer connected to a network. Computer 400 can be a client computer or a server. As shown in FIG. 4, computer 400 can be any suitable type of microprocessor-based device, such as a personal computer, workstation, server, or handheld computing device, such as a phone or tablet. The computer can include, for example, one or more of processor 410, input device 420, output device 430, storage 440, and communication device 460. Input device 420 and output device 430 can correspond to those described above and can either be connectable or integrated with the computer.

Input device 420 can be any suitable device that provides input, such as a touch screen or monitor, keyboard, mouse, or voice-recognition device. Output device 430 can be any suitable device that provides an output, such as a touch screen, monitor, printer, disk drive, or speaker.

Storage 440 can be any suitable device that provides storage, such as an electrical, magnetic, or optical memory, including a random access memory (RAM), cache, hard drive, CD-ROM drive, tape drive, or removable storage disk. Communication device 460 can include any suitable device capable of transmitting and receiving signals over a network, such as a network interface chip or card. The components of the computer can be connected in any suitable manner, such as via a physical bus or wirelessly. Storage 440 can be a non-transitory computer-readable storage medium comprising one or more programs, which, when executed by one or more processors, such as processor 410, cause the one or more processors to execute methods described herein.

Software 450, which can be stored in storage 440 and executed by processor 410, can include, for example, the programming that embodies the functionality of the present disclosure (e.g., as embodied in the systems, computers, servers, and/or devices as described above). In some embodiments, software 450 can include a combination of servers such as application servers and database servers.

Software 450 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include but is not limited to, an electronic, magnetic, optical, electromagnetic, or infrared wired or wireless propagation medium.

Computer 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer 400 can implement any operating system suitable for operating on the network. Software 450 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

1. A system for generating and displaying a medical note, the system comprising one or more processors configured to cause the system to:
display a graphical user interface comprising a plurality of screens;
receive transcript data of a medical consultation;
generate one or more medical indicators based on the received transcript data of the medical consultation;
display, on a first screen of the plurality of screens, a plurality of medical indicators including the one or more medical indicators generated based on the received transcript data;
receive a first user input;
update at least one medical indicator of the plurality of medical indicators displayed on the first screen based at least in part on the received first user input;
generate at least a portion of a medical note based on information indicated by the updated at least one medical indicator; and
display, on a second screen of the plurality of screens, the generated medical note.

2. The system of claim 1, wherein a state of the at least one medical indicator is interchangeable between a plurality of states comprising two or more states of the following: a confirmed present state, a confirmed absent state, a suggested present state, and a suggested absent state.

3. The system of claim 2, wherein updating the at least one medical indicator comprises changing the state of the at least one medical indicator based on the patient medical information.

4. The system of claim 3, wherein the state of the at least one medical indicator is updated to the confirmed present state or confirmed absent state.

5. The system of claim 2, wherein displaying the plurality of medical indicators comprises, in accordance with a given medical indicator of the plurality of medical indicators having been generated based on the received transcript data, displaying the given medical indicator in a state selected from the following: the suggested present state and the suggested absent state.

6. The system of claim 5, wherein the one or more processors are configured to cause the system to, in accordance with not receiving a user input comprising an instruction to update the given medical indicator, generate at least a portion of the medical note to include information indicated by the given medical indicator.

7. The system of claim 1, wherein the one or more processors are configured to cause the system to generate one or more medical indicators included in the displayed plurality of medical indicators based on stored template data;
wherein displaying the plurality of medical indicators comprises, in accordance with a given medical indicator of the plurality of medical indicators having been generated based on the stored template data, displaying the given medical indicator in a state selected from the following: the suggested present state and the suggested absent state.

8. The system of claim 5, wherein the one or more processors are configured to cause the system to, in accordance with not receiving a user input comprising an instruction to update the given medical indicator, generate at least a portion of the medical note without inclusion of information indicated by the given medical indicator.

9. The system of claim 1, wherein updating the at least one medical indicator comprises adding the at least one medical indicator based on the patient medical information.

10. The system of claim 1, wherein the one or more processors are configured to cause the system to automatically organize the plurality of medical indicators displayed on the first screen based on at least complaint type and medical note sections, wherein the medical note sections comprise two or more sections of the following: history of present illness, physical examination, review of systems, and assessment/plan.

11. The system of claim 10, wherein the one or more processors are configured to cause the system to automatically organize the plurality of medical indicators in a given medical note section displayed on the first screen based on one or more sub-sections of the following: medications, symptoms, treatments, tests, and assessments.

12. The system of claim 1, wherein the one or more processors are configured to cause the system to display, on a third screen of the plurality of screens, a representation of the received transcript data.

13. The system of claim 12, wherein the one or more processors are configured to cause the system to indicate one or more of the generated medical indicators in the representation of the transcript data on the third screen.

14. The system of claim 13, wherein indicating the one or more of the generated medical indicators in the representation of the transcript data comprises displaying a portion of text in the representation of the transcript data in a text color that differs from other text in the representation of the transcript data, wherein the portion of text corresponds to the one or more of the generated medical indicators.

15. The system of claim 12, wherein the one or more processors are configured to cause the system to display, on a fourth screen of the plurality of screens, a plurality of graphical representations of respective medical consultations for a medical professional.

16. The system of claim 15, wherein each graphical representation of a medical consultation indicates a respective medical note status for the medical consultation.

17. The system of claim 15, wherein the one or more processors are configured to cause the system to, while displaying the fourth screen, receive a second user input comprising an indication of a selection of a graphical representation of a respective medical consultation of the plurality of graphical representations of respective medical consultations.

18. The system of claim 17, wherein the one or more processors are configured to cause the system to responsively display the first screen of the plurality of screens on the graphical user interface based on the second user input.

19. The system of claim 1, wherein receiving the first user input comprising patient medical information comprises receiving an indication of a selection from a displayed list of options.

20. The system of claim 1, wherein the one or more processors are configured to cause the system to, while displaying the second screen, receive a third user input comprising medical information.

21. The system of claim 20, wherein receiving the third user input comprises one or more of the following: receiving entry of text into one or more text fields, receiving audio dictation, and receiving a selection from a displayed list of options.

22. The system of claim 1, wherein the graphical user interface is embodied in a mobile user interface and at least one of the plurality of screens are touch-sensitive screens.

23. The system of claim 1, wherein the one or more processors are configured to cause the system to store the medical note in an electronic health record (EHR) of a patient.

24. A method for generating and displaying a medical note, the method performed at a system comprising one or more processors, the method comprising:
displaying a graphical user interface comprising a plurality of screens;
receiving transcript data of a medical consultation;
generating one or more medical indicators based on the received transcript data of the medical consultation;
displaying, on a first screen of the plurality of screens, a plurality of medical indicators including the one or more medical indicators generated based on the received transcript data;
receiving a first user input;
updating at least one medical indicator of the plurality of medical indicators displayed on the first screen based at least in part on the received user input;
generating at least a portion of a medical note based on information indicated by the updated at least one medical indicator; and
displaying, on a second screen of the plurality of screens, the generated medical note.

25. A non-transitory computer-readable storage medium storing instructions for generating and displaying a medical note, the instructions configured to be executed by a system comprising one or more processors to cause the system to:
display a graphical user interface comprising a plurality of screens;
receive transcript data of a medical consultation;
generate one or more medical indicators based on the received transcript data of the medical consultation;
display, on a first screen of the plurality of screens, a plurality of medical indicators including the one or more medical indicators generated based on the received transcript data;
receive a first user input;
update at least one medical indicator of the plurality of medical indicators displayed on the first screen based at least in part on the received first user input;
generate at least a portion of a medical note based on information indicated by the updated at least one medical indicator; and
display, on a second screen of the plurality of screens, the generated medical note.
